(19) United States
(12) Patent Application Publication
(10) Pub. No.: US 2025/0082564 A1
(43) Pub. Date: Mar. 13, 2025

(54) OIL-IN-WATER EMULSION COSMETIC

(71) Applicant: SHISEIDO COMPANY, LTD., Chuo-ku, Tokyo (JP)

(72) Inventors: Kotaro TAKADA, Tokyo (JP); Taiki KUMABE, Tokyo (JP); Yoshiro SADAKAMI, Tokyo (JP)

(73) Assignee: SHISEIDO COMPANY, LTD., Chuo-ku, Tokyo (JP)

(21) Appl. No.: 18/294,890

(22) PCT Filed: Aug. 26, 2022
(86) PCT No.: PCT/JP2022/032172

§ 371 Date: Feb. 2, 2024

(30) Foreign Application Priority Data
Sep. 3, 2021 (JP) 2021-143756

(51) Int. Cl. A61K 8/894; A61K 8/02; A61K 8/06; A61Q 1/02

(52) U.S. Cl. CPC A61K 8/894; A61K 8/0254; A61K 8/062; A61Q 1/02; A61K 2800/43

(57) ABSTRACT

[Problem] An objective of the present invention is to provide an oil-in-water emulsion cosmetic having a watery and well-spreading texture in use and having excellent covering performance.

[Solution] The oil-in-water emulsion cosmetic of the present invention contains (A) an aqueous component selected from monohydric alcohols and dihydric alcohols; (B) a polyoxyalkylene-modified silicone; (C) an oil; and (D) a pigment having a hydrophobized surface; wherein the (A) aqueous component, in the case of being a monohydric alcohol alone, constitutes 1% to 15% by mass relative to the overall amount of the cosmetic, in the case of being a dihydric alcohol alone, constitutes 1% to 20% by mass relative to the overall amount of the cosmetic, and in the case of being a combination of a monohydric alcohol and a dihydric alcohol, a total amount thereof constitutes 1% to 45% by mass relative to the overall amount of the cosmetic.

### TECHNICAL FIELD

The present invention relates to an oil-in-water emulsion cosmetic having a watery and well-spreading texture in use.

### BACKGROUND ART

Generally, water-in-oil emulsions are favorably used when preparing makeup cosmetics since they allow large quantities of emollients and powder components to be blended as oil-based components. Water-in-oil emulsions allow high cosmetic effects to be obtained by blending powder components such as pigments, yet there are cases in which they perform poorly in terms of having a watery and fresh texture in use or having good spreadability when applied, like skin-care cosmetics.

Meanwhile, oil-in-water emulsions are excellent in terms of a watery texture in use and good spreadability when applied, and are therefore sometimes used not only in skin-care cosmetics, but also in makeup cosmetics. However, when large quantities of powder components are to be blended in order to cover skin roughness and blemishes such as spots and freckles, there were problems in that the feel in use was reduced, such as the wateriness becoming worse and powderiness occurring.

As technology for overcoming the above-mentioned problems, for example, Patent Document 1 provides an oil-in-water emulsion cosmetic with an improved texture in use due to thickening the water phase, which is the external phase, by blending an associative thickener and a thickening polysaccharide, and blending a specific dispersant and a powder into the oil phase, which is the internal phase.

In the above-mentioned oil-in-water emulsion cosmetic, a resilient and smooth texture in use is obtained by thickening the water phase. However, the performance could not be considered to be sufficient in terms of the watery texture in use and the spreadability when applied, which are characteristic of oil-in-water emulsions.

### RELATED ART

### Patent Documents

Patent Document 1: JP 2009-234917 A

### SUMMARY OF THE INVENTION

### Problem to be Solved by the Invention

An objective of the present invention is to provide an oil-in-water emulsion cosmetic having a watery and well-spreading texture in use, even when blending large quantities of a pigment for providing excellent covering performance in terms of cosmetic effects.

### Means for Solving the Problem

The present inventors performed diligent research towards solving the aforementioned problem, as a result of which they discovered that a texture in use characteristic of oil-in-water emulsions can be obtained, even when blending a pigment into the oil phase, by emulsifying an oil-in-water emulsion cosmetic by silicone nanodiscs having, as precursors, vesicles formed by a specific aqueous component and a specific silicone-based surfactant, thereby completing the present invention.

That is, the present invention provides an oil-in-water emulsion cosmetic containing:
(A) an aqueous component selected from monohydric alcohols and dihydric alcohols; (B) a polyoxyalkylene-modified silicone;
(C) an oil; and
(D) a pigment having a hydrophobized surface;
wherein the (A) aqueous component, in the case of being a monohydric alcohol alone, constitutes 1% to 15% by mass relative to the overall amount of the cosmetic, in the case of being a dihydric alcohol alone, constitutes 1% to 20% by mass relative to the overall amount of the cosmetic, and in the case of being a combination of a monohydric alcohol and a dihydric alcohol, a total amount thereof constitutes 1% to 45% by mass relative to the overall amount of the cosmetic.

### Effects of the Invention

The cosmetic of the present invention, due to the above-mentioned features, can provide a texture in use that is watery, that has excellent spreadability when applied, and that does not have powderiness. According to the present invention, a pigment can be blended into the oil phase, which is the internal phase, and therefore, covering performance can be provided as a cosmetic effect. In the cosmetic of the present invention, since the amount of the silicone oil can be set to be 50% by mass or more, a dry feel in use with little stickiness can be obtained. Additionally, the powder dispersibility can be improved by blending an (E) oil-phase thickener in addition to components (A) to (D) above.

### MODES FOR CARRYING OUT THE INVENTION

The oil-in-water emulsion cosmetic according to the present invention is characterized by containing (A) an aqueous component selected from monohydric alcohols and dihydric alcohols, (B) a polyoxyalkylene-modified silicone, (C) an oil, and (D) a pigment having a hydrophobized surface. The respective components constituting the cosmetic of the present invention will be explained in detail below.

### <(A) Aqueous Component>

The (A) aqueous component (hereinafter sometimes referred to simply as “component (A)”) blended into the cosmetic according to the present invention refers to one or more components selected from among monohydric alcohols and dihydric alcohols.

The monohydric alcohols, though not particularly limited as long as they are normally used in cosmetics, include ethyl alcohol (ethanol), modified alcohols, normal propyl alcohol, isopropyl alcohol, etc. In the present invention, ethyl alcohol is preferred.

The dihydric alcohols, though not particularly limited as long as they are normally used in cosmetics, include 1,3-butylene glycol, dipropylene glycol, etc. In the present invention, dipropylene glycol is preferred.

The spherical vesicles formed by the surfactant have surfaces that are entirely covered by hydrophilic groups. However, since the nanodiscs have lipophilic groups at edge portions thereof, it is difficult to form the nanodiscs in water. If monohydric and dihydric alcohols are present in the water, the surfactant (the polyoxyalkylene-modified silicone in the present invention) is hydrophilized by solvent effects, and as a result thereof, a transition from the spherical vesicles to nanodiscs is promoted.

Meanwhile, when dissolving a polyoxyalkylene-modified silicone such as PEG-12 dimethicone in alcohol, there is a tendency for trihydric alcohols such as glycerin and polyhydric alcohols such as sorbitol to lipophilize the surfactant and to inhibit the transition to nanodiscs. Therefore, in the case in which a trihydric or higher alcohol is to be blended, the total amount of monohydric and dihydric alcohols is preferably made larger than the total amount of trihydric or higher polyhydric alcohols.

When using a monohydric alcohol alone, the blended amount thereof should be 1% to 15% by mass relative to the overall amount of the cosmetic, and when using a dihydric alcohol alone, the blended amount thereof should be 1% to 20% by mass relative to the overall amount of the cosmetic. Additionally, when using a combination of a monohydric alcohol and a dihydric alcohol, the total blended amount thereof should be 1% to 45% by mass, preferably 1% to 35% by mass, relative to the overall amount of the cosmetic. More preferably, they should be blended so that the upper limits of the concentrations of the monohydric alcohol and the dihydric alcohol satisfy expression (1) below.

Water-phase monohydric alcohol concentration (%)/15+water-phase dihydric alcohol concentration (% by mass)/20≤1 (1)

If the blended amount of the monohydric alcohol alone, the blended amount of the dihydric alcohol alone, or the total blended amount of the monohydric alcohol and the dihydric alcohol is less than 1% by mass, then there are cases in which vesicles are not formed or the structure becomes disturbed and emulsification is not possible. Additionally, in the case in which the blended amount of the monohydric alcohol alone exceeds 15% by mass, in the case in which the blended amount of the dihydric alcohol alone exceeds 20% by mass, in the case in which the blending ratio between the monohydric alcohol and the dihydric alcohol lies outside the range in Expression (1) above, or even in the case in which the blending ratio is within the range in Expression (1) above, if the total blended amount exceeds 45% by mass, there are cases in which the vesicle membranes become too flexible or the vesicles transition to micelles, so that stabilization effects cannot be obtained.

### <(B) Polyoxyalkylene-Modified Silicone>

The (B) polyoxyalkylene-modified silicone (hereinafter sometimes referred to simply as “component (B)”) blended into the cosmetic of the present invention is a surfactant having polysiloxane structures as hydrophobic groups and polyoxyalkylene structures as hydrophilic groups, and is preferably a water-soluble silicone-based surfactant in which some of the methyl groups in the dimethicone are substituted by polyethylene glycol. Said component is specifically represented by the following expression (2).

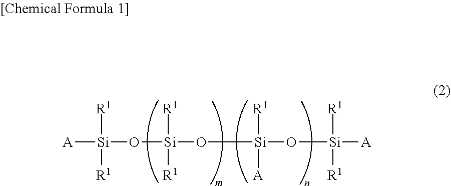

In the aforementioned expression (2), R¹ represents hydrogen or an alkyl group with 1 to 6 carbon atoms, which may be the same or independently different. At least one of the groups A is a polyoxyalkylene group represented by expression (3):

—(CH₂)a-(C₂H₄O)b-(C₃H₆O)c-R² (3)

The other groups A may each be a hydrogen or an alkyl group with 1 to 6 carbon atoms, which may be the same or independently different. In expression (3), R² represents hydrogen or an alkyl group with 1 to 6 carbon atoms, a is an integer from 1 to 6, b is an integer from 0 to 50, and c is an integer from 0 to 50, where b+c is at least 5 or larger. In the aforementioned expression (2), m is an integer from 1 to 200, and n is an integer from 0 to 50.

The (B) polyoxyalkylene-modified silicone in the present invention preferably has an HLB of less than 10 by an HLB calculation based on the Griffin formula.

In the cosmetic according to the present invention, the (B) polyoxyalkylene-modified silicone is preferably, in particular, a PEG-12 dimethicone according to the aforementioned expression (3), where c is 0 and b is 12. Additionally, the PEG-12 dimethicone more preferably has an HLB of less than 10.

Examples of commercially available products that are PEG-12 dimethicones include DOWSIL™ ES-5373, SH3772M, SH3773M, SH3775M (all manufactured by Dow Toray Co., Ltd.), IM-22 (manufactured by Wacker Chemical Corp.), etc.

The blended amount of component (B) is not particularly limited as long as vesicles, which are nanodisc precursors, can be formed, and may, for example, be 0.1% to 5.0% by mass, preferably 0.3% to 3.0% by mass, and more preferably 0.8% to 2.0% by mass relative to the overall amount of the cosmetic. If the blended amount is less than 0.1% by mass, then there are cases in which vesicles are not sufficiently formed, and if the blended amount exceeds 5.0% by mass, there are cases in which the vesicle stability is poor.

In the present invention, a vesicle refers to a spherical closed body consisting of a bimolecular membrane (lamellar liquid crystal). The cosmetic according to the present invention contains nanodiscs comprising the surfactant of component (B). The vesicles, which are precursors to the nanodiscs, can be formed from a conventional method. Specifically, the vesicles comprising component (B) are formed by mixing and stirring the (A) aqueous component and the (B) polyoxyalkylene-modified silicone. During vesicle formation, in addition to the (A) aqueous component, water and aqueous components normally used in cosmetic products may be blended in an amount within a range not compromising the stability of the vesicles. The average particle size of the vesicles is not particularly limited, but should normally be approximately 30 nm to 150 nm.

The “nanodiscs” mentioned here are plate-shaped lamellar liquid-crystal closed bodies with vesicles (lamellar liquid-crystal spherical closed bodies) formed from amphiphilic substances as precursors, not enclosing water-soluble components in the interiors of the closed bodies, and having lipophilic groups at edge portions. The nanodiscs are present as vesicles, which are the precursors, in compositions not containing oils, and undergo a structural change (hereinafter also referred to as a “transition”) to nanodiscs when an oil is added and emulsification is performed. The nanosdiscs of the present invention are obtained by mixing a polyoxyalkylene-modified silicone with an aqueous component selected from monohydric alcohols and dihydric alcohols and forming vesicles to obtain an aqueous vesicle dispersion, then adding an anionic surfactant and an oil to the aqueous vesicle dispersion and dispersing the components while applying a strong stirring force. In the emulsified state, the nanodiscs are present in a state of adsorption to the oil/water interface and contribute to emulsion stability. In the present description, the amphiphilic substance forming the vesicles is a silicone-based surfactant. Thus, the nanodiscs of the present invention will also be referred to as “silicone nanodiscs”.

### <(C) Oil>

The (C) oil (hereinafter sometimes referred to simply as “component (C)”) blended into the cosmetic according to the present invention is not particularly limited as long as it is an oil that is normally blended into cosmetics, and examples include hydrocarbon oils, silicone oils, ester oils, higher alcohols having 12 to 22 carbon atoms, etc.

Hydrocarbon oils include, for example, liquid paraffin, isohexadecane, isododecane, ozokerite, squalane, squalene, pristane, paraffin, isoparaffin, ceresin, vaseline, hydrogenated polyisobutene, olefin oligomers, volatile hydrocarbon oils (for example, isododecane, isohexadecane, undecane, tridecane, etc.), etc.

Silicone oils include, for example, linear polysiloxanes (for example, dimethicone, diphenylsiloxy phenyl trimethicone, diphenyl dimethicone, etc.), cyclic polysiloxanes (for example, octamethyl cyclotetrasiloxane, decamethyl cyclopentasiloxane, dodecamethyl cyclohexasiloxane, etc.), silicone resins forming three-dimensional reticulated structures, silicone rubbers, various types of modified polysiloxanes (amino-modified polysiloxane, polyether-modified polysiloxane, alkyl-modified polysiloxane, fluorine-modified polysiloxane, etc., except that those having oil-phase thickening effects are excluded), alkyl silicones, etc.

Ester oils include, for example, ethylhexyl methoxycinnamate, pentaerythrityl tetraethylhexanoate, cetyl ethylhexanoate, jojoba oil, di(phytosteryl/octyldodecyl) lauroyl glutamate, triisostearin, glyceryl diisostearate, triethyl hexanoin, (phytosteryl/behenyl) dimer dilinoleate, (phytosteryl/isostearyl/cetyl/stearyl/isostearyl/cetyl/stearyl/behenyl) dimer dilinoleate, isopropyl palmitate, phytosteryl macadamiate, pentaerythrityl tetra(behenate/benzoate/ethylhexanoate), ethylhexyl palmitate, myristyl myristate, isopropyl myristate, tripropylene glycol dipivalate, diisopropyl sebacate, isodecyl neopentanoate, octyl octanoate, nonyl nonanoate, cetyl octanoate, octyldodecyl myristate, butyl stearate, hexyl laurate, decyl oleate, hexyldecyl dimethyl octanoate, cetyl lactate, myristyl lactate, lanolin acetate, isocetyl stearate, isocetyl isostearate, cholesteryl 12-hydroxystearate, ethylene glycol di-2-ethylhexanoate, dipentaerythritol fatty acid ester, N-alkylglycol monoisostearate, neopentyl glycol dicaprate, tripropylene glcol pivalate, diisotearyl malate, glycerin di-2-heptylundecanoate, glycerin diisostearate, trimethylolpropane tri-2-ethylhexanoate, trimethylolpropane triisostearate, pentaerythritol tetra-2-ethylhexanoate, glycerin tri-2-ethylhexanoate, glycerin trioctanoate, glycerin triisoplamitate, cetyl 2-ethylhexanoate-2-ethylhexyl palmitate, glycerin trimyristate, glyceride tri-2-heptylundecanoate, castor oil fatty acid methyl ester, oleyl oleate, acetoglyceride, 2-heptylundecyl palmitate, diisobutyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, 2-ethylhexyl succinate, triethyl citrate, etc.

Higher alcohols having 12 to 22 carbon atoms include, for example, oleyl alcohol, 2-decyltetradecinol, dodecanol, isostearyl alcohol, octyldodecanol, etc.

In the present invention, the percentage of the total blended amount of the (C) oil that is occupied by silicone oils may be 50% or higher in terms of the percentage by mass, in order to further improve the properties in use of the cosmetic. By setting the percentage of the total blended amount of the (C) oil that is occupied by silicone oils to be high, a dry feel in use with little stickiness can be obtained in a cosmetic in which a powder is blended in the oil phase.

Additionally, in the cosmetic of the present invention, the percentage of the total blended amount of the (C) oil that is occupied by the volatile silicone oil should preferably be 25% or lower in terms of percentage by mass for the purpose of improving the powder dispersibility. Since the cosmetic according to the present invention does not need to have a volatile silicone oil blended therein as the (C) oil, the lower limit of the percentage of the total blended amount of the (C) oil that is occupied by the volatile silicone oil is 0%.

In the present invention, volatile silicone oils refer to silicone oils having a volatilization rate, at 25° C., such that there is an hourly weight change of 30% or more. In this case, the volatilization rate refers to the value of the hourly weight change measured under 25° C. conditions by a weighing method after placing filter paper on a glass Petri dish and dripping approximately 0.2 g of a sample thereon. Specific examples of volatile silicone oils include isododecane, low-viscosity volatile silicones (low-viscosity dimethicones) having a degree of polymerization lower than 650, etc. Commercially available products that are volatile silicone oils include Creasil ID CG (manufactured by Shima Trading Co., Ltd.), KF-96L-1.5CS (manufactured by Shin-Etsu Chemical Co., Ltd.), etc. The volatilization rate of Creasil ID CG is 90% or higher, and the volatilization rate of KF-96L-1.5CS is approximately 50%.

The blended amount of the (C) oil blended into the cosmetic according to the present invention is not particularly limited as long as it is an amount that is normally used when blending a powder into an oil phase, and may, for example, be 1% to 40% by mass relative to the overall amount of the cosmetic. The stability and the properties in use tend to become worse when the blended amount of the (C) oil exceeds 40% by mass.

### <(D) Pigment Having Hydrophobic Surface>

The (D) pigment having a hydrophobic surface (hereinafter sometimes referred to simply as “component (D)”) blended into the cosmetic according to the present invention refers to a pigment that is generally used in cosmetics, in which the particle surfaces are hydrophobic. The pigment of the present invention preferably has an average particle size of 100 nm to 1 m. The component (D) in the present invention may be either non-surface-treated or subjected to a hydrophobization treatment, as long as the particle surface is hydrophobic.

Specific examples of pigments include inorganic powders such as talc, kaolin, mica, sericite, muscovite, phlogopite, synthetic mica, lepidolite, biotite, lithia mica, vermiculite, magnesium carbonate, calcium carbonate, aluminum silicate, barium silicate, calcium silicate, magnesium silicate, strontium silicate, tungstic acid metal salts, magnesium, silica, zeolite, barium sulfate, calcined calcium sulfate (calcined plaster), calcium phosphate, fluorapatite, hydroxyapatite, ceramic powder, metal soaps (zinc myristate, calcium palmitate, aluminum stearate, etc.) and boron nitride; inorganic white pigments such as titanium dioxide and zinc oxide; inorganic red pigments such as iron oxide (red iron oxide) and iron titanate; inorganic brown pigments such as γ-iron oxide; inorganic yellow pigments such as yellow iron oxide and ocher; inorganic black pigments such as black iron oxide, carbon black and lower titanium oxides; inorganic violet pigments such as mango violet and cobalt violet; inorganic green pigments such as chromium oxide, chromium hydroxide and cobalt titanate; inorganic blue pigments such as ultramarine blue and Prussian blue; pearlescent pigments such as titanium dioxide-coated mica, titanium dioxide-coated bismuth oxychloride, titanium dioxide-coated talc, colored titanium dioxide-coated mica, bismuth oxychloride and argentine; and metallic powder pigments such as aluminum powder and copper powder.

In particular, examples include iron oxide (red iron oxide), iron titanate, γ-iron oxide, yellow iron oxide, ocher, black iron oxide, carbon black, lower titanium oxides, mango violet, cobalt violet, chromium oxide, chromium hydroxide, cobalt titanate, ultramarine blue, Prussian blue, etc. Furthermore, the pigment in the present invention is preferably a pigment-grade iron oxide such as yellow iron oxide, red iron oxide and black iron oxide, a pigment-grade titanium oxide, etc.

The hydrophobization surface treatment agents include silicone treatment agents, fluorine compound treatment agents, amino acid treatment agents, fatty acid treatment agents, fatty acid soap treatment agents, fatty acid ester treatment agents, lecithin treatment agents, alkyl phosphoric acid ester treatment agents, etc. In the present invention, the powder is preferably hydrophobized with a surface treatment agent containing an amino acid or a salt thereof for the purpose of improving the powder dispersibility.

Examples of the “amino acid” used as the surface treatment agent include glutamic acid, aspartic acid, lysine etc., among which glutamic acid and aspartic acid are preferred. Additionally, the “amino acid” may be an “amino acid acylated by a saturated fatty acid” in which an acyl group, preferably a saturated fatty acid having 12 to 20 carbon atoms, is condensed on an amino group in the amino acid. Examples of the “acyl group” include stearoyl groups, lauroyl groups, etc. The “salt” may be selected from among alkali metal salts such as sodium and potassium, and alkaline earth metal salts, among which sodium salts are preferred. Specific examples of acylated amino acids include disodium stearoyl glutamate, sodium lauroyl glutamate and sodium lauroyl aspartate.

Component (D) in the present invention can be prepared by a conventional method, by adsorbing an amino acid or a salt thereof to the powder surfaces.

The aforementioned amino acids or salts thereof may be solely used as the surface treatment agent of component (D) in the present invention, or another compound that is generally used as a surface treatment agent for powders may be further included in addition to the aforementioned amino acid or a salt thereof.

A commercially available product may be used as component (D) in the present invention, and preferable commercially available products include, for example, ASL-treated powders such as ASL-1 TiO2 CR-50, ASL-Red R516P, ASL-Yellow LL-100P and ASL-Black BL-100P; ASI-treated powders such as ASI TiO2 CR-50, ASI-Red R516P, ASI-Yellow LL-100P, ASI-Black BL-100P and ASI-Talc JA46R (all of the above manufactured by Daito Kasei Kogyo Co., Ltd.); and NHS-treated powders such as NHS-Titanium CR-50, NHS-Red R516PS, NHS-Yellow LL-100P, NHS-Black BL-100P, NHS-Mica M-102 and NHS-Talc JA-46R (all of the above manufactured by Miyoshi Kasei, Inc.).

The blended amount of the aforementioned component (D) is not particularly limited, and in order to obtain the desired covering performance, the lower limit value relative to the overall amount of the cosmetic may be 4% by mass or more, 5% by mass or more, or 6% by mass or more, and the upper limit value may be 30% by mass or less, 25% by mass or less, or 20% by mass or less. The blending range is, for example, 4% to 30% by mass, and preferably 6% to 20% by mass. If the blended amount is less than 4% by mass, then there are cases in which sufficient covering performance cannot be obtained, and if the blended amount exceeds 30% by mass, then the stability tends to become poor.

The cosmetic according to the present invention is a powder-in-oil-in-water type composition in which component (D) is dispersed in oil droplets, which are the internal phase.

In the cosmetic according to the present invention, the oil phase is preferably 1% to 50% by mass, more preferably 1% to 30% by mass relative to the overall amount of the cosmetic.

The dispersibility of the powder components blended in the oil phase can be improved by blending an (E) oil-phase thickener (hereinafter sometimes referred to simply as “component (E)”) into the cosmetic according to the present invention in addition to the aforementioned components (A) to (D).

### <(E) Oil-Phase Thickener>

The (E) oil-phase thickener (hereinafter sometimes referred to simply as “component (E)”) blended into the cosmetic according to the present invention can be appropriately selected from among substances used as components for achieving effects of thickening an oil phase by dissolving into an oil or swelling with the oil in normal emulsion cosmetics, etc. For example, there are organically modified clay minerals, waxes, dextrin fatty acid esters, glyceryl fatty acid esters, amino acid gelling agents, sucrose fatty acid esters, solid or semi-solid hydrocarbon oils, fatty acids or salts thereof, or polyether-modified silicones other than the aforementioned component (B), etc.

Organically modified clay minerals are a type of colloidal aluminum hydrosilicate having a three-layered structure, among which the clay mineral represented by general formula (4) below, modified by a quaternary ammonium salt-type cationic surfactant, is typical.

(X,Y)₂₋₃(Si,Al)₄O₁₀(OH)₂Z_{1/3}·nH₂O (4)

(In the formula above, X=Al, Fe(III), Mn(III) or Cr(III); Y=Mg, Fe(II), Ni, Zn or Li; and Z=K, Na or Ca)

Specific examples of organically modified clay minerals include dimethyldistearyl ammonium hectorite (disteardimonium hectorite), dimethylalkyl ammonium hectorite, benzyldimethylstearyl ammonium hectorite, distearyldimethyl ammonium chloride-treated aluminum-magnesium silicate, etc. As commercially available products, Bentone 27 (benzyldimethylstearyl ammonium chloride-treated hectorite, manufactured by Elementis Japan) and Bentone 38 (distearyldimethyl ammonium chloride-treated hectorite, manufactured by Elementis Japan) are preferable.

The waxes are not particularly limited as long as they are in solid or paste form at 25° C. and are generally used in cosmetics, and examples include highly polymerized methyl polysiloxanes such as highly polymerized dimethyl polysiloxane, highly polymerized methylphenyl siloxane and highly polymerized methylvinyl polysiloxane, highly polymerized amino-modified methyl polysiloxane, alkyl-modified silicones (for example, stearyl dimethicone, alkyl (C30-C45) methicone, etc.), polyamide-modified silicones and long-chain alkoxy-modified silanes (stearoxytrimethyl silane, etc.), from which one or more types that have been selected may be used, as needed.

Commercially available products that are silicone waxes include silicone wax AMS-C30 (alkyl (C30-C45) methicone, manufactured by Dow Chemical Japan/Dow Toray Co., Ltd.).

Dextrin fatty acid esters are esters of dextrin or reduced dextrin with a higher fatty acid, which can be used without any particular limitations as long as they are generally used in cosmetics. As the dextrin or reduced dextrin, one having an average degree of sugar polymerization of 3 to 100 is preferable. Additionally, as the constituent fatty acid in the dextrin fatty acid ester, a saturated fatty acid having 8 to 22 carbon atoms is preferably used. Specific examples include dextrin palmitate, dextrin oleate, dextrin stearate, dextrin myristate, dextrin (palmitate/2-ethylhexanoate), etc.

Glyceryl fatty acid esters are esterification reaction products obtained by reacting glycerin, a dibasic acid with 18 to 28 carbon atoms, and a fatty acid having 8 to 28 carbon atoms (except that dibasic acids are excluded), which can be used without any particular limitations as long as they are generally used in cosmetics. Specific examples include glyceryl (behenate/isostearate/eicosanedioate), glyceryl (behenate/eicosanedioate), polyglyceryl-10 (behenate/eicosanedioate), etc.

Examples of amino acid gelling agents include N-lauroyl-L-glutamic acid dibutylamide (dibutyl lauroyl glutamide), N-2-ethylhexanoyl-L-glutamic acid dibutylamide (dibutyl ethylhexanoyl glutamide), polyamide-8, polyamide-3, etc.

As sucrose fatty acid esters, those in which the fatty acids therein are linear or branched, saturated or unsaturated, having 12 to 22 carbon atoms, can be preferably used. Specific examples include sucrose caprylic acid esters, sucrose capric acid esters, sucrose lauric acid esters, sucrose myristic acid esters, sucrose palmitic acid esters, sucrose stearic acid esters, sucrose oleic acid esters, sucrose erucic acid esters, etc.

The solid or semi-solid hydrocarbon oils are hydrocarbons that are solid or semi-solid at ambient temperature (25° C.), specific examples including vaseline, hydrogenated palm oil, hydrogenated castor oil (castor wax), hardened palm kernel oil, hardened castor oil, hydrogenated peanut oil, hydrogenated rapeseed oil, hydrogenated camellia oil, hydrogenated soybean oil, hydrogenated olive oil, hydrogenated macadamia nut oil, hydrogenated sunflower oil, hydrogenated wheat germ oil, hydrogenated rice germ oil, hydrogenated rice bran oil, hydrogenated cottonseed oil, hydrogenated avocado oil, waxes and the like.

As the fatty acids, those that are solid at ambient temperature can be used, including, for example, myristic acid, palmitic acid, stearic acid, behenic acid, 12-hydroxystearic acid, etc. Additionally, examples of fatty acid salts include calcium salts, magnesium salts, aluminum salts, etc. of the above.

As the polyether-modified silicones other than the aforementioned component (B), there are PEG/PPG-19/19 dimethicone, etc.

In particular, as the (E) oil-phase thickener in the present invention, one or more types selected from among organically modified clay minerals, dextrin fatty acid esters and polyether-modified silicones other than the aforementioned component (B) is preferably used.

As the (E) oil-phase thickener in the present invention, one type selected from among the above-mentioned substances may be used alone, or a combination of two or more types may be used.

The blended amount of the aforementioned component (E) is not particularly limited as long as the effects of thickening the oil phase is achieved. For example, the lower limit value of the blended amount may be 0.00001% by mass, 0.00003% by mass, 0.00005% by mass, 0.0001% by mass, 0.001% by mass, 0.01% by mass, 0.1% by mass, 0.3% by mass, etc. relative to the overall amount of the cosmetic, and the upper limit value of the blended amount may be 5.0% by mass, 4.0% by mass, 3.0% by mass, 2.0% by mass, etc. For example, the blended amount range may be set to be 0.1% to 5.0% by mass, 0.3% to 3.0% by mass, 0.8% to 2.0% by mass, etc. relative to the overall amount of the cosmetic. If the blended amount exceeds 5.0% by mass, the properties in use and the stability tend to become worse.

In the present invention, the percentage of the total blended amount of the (C) oil that is occupied by silicone oils may be increased in order to further improve the properties in use as a cosmetic. However, if the percentage occupied by silicone oils is increased, then there are cases in which the stability of the system becomes lower, the dispersibility of the powder over time becomes poor and color streaking (streaking that occurs due to the pigments becoming non-uniform) occurs. Since the powder dispersibility increases due to blending the (E) oil-phase thickener in addition to the components (A) to (D) in the cosmetic of the present invention, color streaking that occurs over time can be suppressed even when the percentage of the total blended amount of the (C) oil that is occupied by silicone oils is increased.

In the cosmetic according to the present invention, the emulsion stability can be further improved by blending an (F) anionic surfactant (hereinafter sometimes referred to simply as “component (F)”) in addition to the aforementioned components (A) to (D), or in addition to the aforementioned components (A) to (E).

The (F) anionic surfactant blended into the cosmetic according to the present invention may be of any type that is normally used in cosmetics, and refers to a surfactant, other than a silicone-based surfactant such as the aforementioned (B) polyoxyalkylene-modified surfactants, etc., having an anionic hydrophilic group such as one having a carboxylic acid, sulfonic acid or phosphoric acid structure. The nanodiscs are stabilized by blending the anionic surfactant.

In particular, it is preferable to use an anionic surfactant having a Krafft point higher than room temperature. In the case in which the Krafft point of the anionic surfactant is lower than room temperature, silicone-based surfactants and anionic surfactants easily mix and interact, thereby tending to prevent the transition from vesicles to nanodiscs.

The (F) anionic surfactant blended into the cosmetic according to the present invention is preferably a sulfonic acid salt-type anionic surfactant. Sulfonic acid salt-type anionic surfactants include sulfosuccinic acid diester salts, alkylallyl sulfonic acid salts, alkyl ether sulfonic acid salts, sulfosuccinic acid ester salts, acyl methyltaurine salts, acyl taurine salts, potassium cetyl phosphate, potassium cocoyl glutamate, etc. In particular, it is preferable to be selected from among acyl methyltaurine salts, potassium cetyl phosphate and potassium cocoyl glutamate.

In the present invention, it is particularly preferable to blend an N-acyl methyltaurine salt as the anionic surfactant. Furthermore, an N-stearoyl-N-methyltaurine salt is particularly preferable among the N-acyl methyltaurine salts represented by formula (5) below.

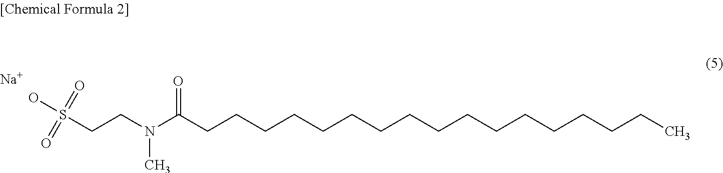 [SMILES: [Na+] ; CCCCCCCCCCCCCCCCCC(=O)N(C)CCS(=O)(=O)O]

Component (F) is an optional component and therefore does not need to be blended. However, when blended, the amount should preferably be 0.001% to 1% by mass, more preferably 0.005% to 0.1% by mass, and even more preferably 0.008% to 0.06% by mass relative to the overall amount of the cosmetic. For example, the blended amount range may be 0.01% to 1% by mass, 0.01% to 0.1% by mass, 0.01% to 0.06% by mass, etc. relative to the overall amount of the cosmetic. If the blended amount is less than 0.001% by mass, then there are cases in which the nanodiscs are not sufficiently stable, and if the blended amount exceeds 1% by mass, then there are cases in which the vesicles, which are nanodisc precursors, are solubilized, or in which the formation of nanodiscs is prevented.

Additionally, when blending the component (F), the blended amount ratio between the (B) polyoxyalkylene-modified silicone and the (F) anionic surfactant is preferably 1:0.01 to 1:0.06 in terms of percentage by mass.

In the cosmetic of the present invention, a (G) dispersant (hereinafter sometimes referred to simply as “component (G)”) may be blended into the oil phase for the purpose of further improving the emulsion stability and the texture in use.

The (G) dispersant blended into the cosmetic according to the present invention may include polyglycerin fatty acid esters having three or more glycerin molecules, long-chain fatty acids, polyhydroxystearic acids, etc.

Specific examples of polyglycerin fatty acid esters having three or more glycerin molecules include polyglyceryl-3 diisostearate, polyglyercyl-3 distearate, polyglyceryl-4 caprate, polyglyceryl-4 stearate, polyglyceryl-4 oleate, polyglyceryl-4 tristearate, polyglyceryl-4 pentaoleate, polyglyceryl-6 laurate, polyglyceryl-6 myristate, polyglyceryl-6-stearate, polyglyceryl-6 oleate, polyglyceryl-6 caprate, polyglyceryl-6 dicaprate, polyglyceryl-6 dioleate, polyglyceryl-6 tristearate, polyglyceryl-6 tetrabehenate, polyglyceryl-6 pentastearate, polyglyceryl-6 pentaoleate, polyglyceryl-6 polyricinoleate, polyglyceryl-6 polyhydroxystearate, polyglyceryl-10 laurate, polyglyceryl-10 myristate, polyglyceryl-10 stearate, polyglyceryl-10 isostearate, polyglyceryl-10 oleate, polyglyceryl-10 linoleate, polyglyceryl-10 distearate, polyglyceryl-10 diisostearate, polyglyceryl-10 tristearate, polyglyceryl-10 trioleate, polyglyceryl-10 pentastearate, polyglyceryl-10 pentahydroxystearate, polyglyceryl-10 pentaisostearate, polyglyceryl-10 pentaoleate, polyglyceryl-10 heptastearate, polyglyceryl-10 heptaoleate, polyglyceryl-10 decastearate, polyglyceryl-10 decaisostearate, polyglyceryl-10 decaoleate, polyglyceryl-10 decamacadamiate, polyglyceryl-10 polyricinoleate, etc. In particular, it is preferable to use polyglyceryl-6 polyricinoleate and polyglyceryl-6 polyhydroxystearate.

Specific examples of long-chain fatty acids include isostearic acid, etc.

Polyhydroxystearic acids are compounds obtained by hydroxystearic acid being oligomerized by forming ester bonds, and are not particularly limited as long as they are generally used in cosmetics. Specific examples of polyhydroxystearic acids, though not particularly limiting, include those, for example, with a degree of polymerization of 4 to 8.

The (G) dispersant is an optional blended component in the cosmetic of the present invention and therefore does not necessarily need to be blended. However, when blended, it should be blended at a level at which the effects of said blending can be observed, within limits in which adverse effects such as the blended amount being excessive and thereby degrading the feel in use are not observed. As a suitable blended amount of the dispersant in the cosmetic according to the present invention, it should preferably be approximately 0.1% to 10% by mass relative to the overall amount of the cosmetic.

A water-phase thickener may be blended into the cosmetic according to the present invention for the purpose of further improving the emulsion stability. The water-phase thickener is not particularly limited as long as it is normally used in cosmetics. Examples include plant-based polymers such as gum arabic, tragacanth gum, galactan, guar gum, carrageenan, pectin, quinceseed (marmelo) extract, brown algae powder and agar; microbe-based polymers such as hyaluronic acid, sodium hyaluronate, xanthan gum, dextran and pullulan; starches such as starch, carboxymethyl starch and methyl hydroxy starch; celluloses such as methyl cellulose, nitrocellulose, ethyl cellulose, methyl hydroxypropyl cellulose, hydroxyethyl cellulose, cellulose sulfuric acid salts, hydroxypropyl cellulose, carboxymethyl cellulose, crystalline cellulose and cellulose powder; vinyl-based polymers such as polyvinyl alcohol, polyvinyl methyl ether, polyvinyl pyrrolidone and carboxyvinyl polymer; acrylic-based polymers such as polyacrylic acid and salts thereof, and polyacrylimides; taurate-based polymers such as (dimethyl acrylamide/sodium acryloyl dimethyl taurate) crosspolymer, (ammonium acryloyl dimethyl taurate/vinyl pyrrolidone) copolymer and (ammonium acryloyl dimethyl taurate/beheneth-25 methacrylate) crosspolymer; acrylate-based polymers such as alkyl acrylate/methacrylate copolymers such as acrylates/steareth-20 methacrylate copolymer, and acrylates/alkyl acrylate crosspolymers such as acrylates/alkyl (C10-30) acrylate crosspolymer; hydrophobically modified polyether urethanes such as PEG-240/decyltetradeceth-20/hexamethylene diisocyanate copolymer; and glycyrrhizic acid, alginic acid and salts thereof, etc.

The water-phase thickener is an optional blended component in the cosmetic of the present invention and therefore does not necessarily need to be blended. When blended, it should be blended at a level at which the effects of said blending can be observed, within limits in which adverse effects such as the blended amount being excessive and thereby degrading the feel in use are not observed. As a suitable blended amount of the water-soluble thickener in the cosmetic according to the present invention, it should preferably be approximately 0.1% to 10% by mass relative to the overall amount of the cosmetic.

The water blended into the cosmetic according to the present invention is selected, as needed, from among ion-exchanged water, purified water, tap water, natural water, etc. The blended amount is the balance (in percentage by mass relative to the overall amount of the cosmetic) relative to the sum of the essential components and other optional blended components in the present invention. Generally, approximately 30% to 70% by mass relative to the overall amount of the cosmetic is suitable.

Aside from the components mentioned above, other optional additive components that are normally used in external preparations for the skin, such as cosmetic products and pharmaceutical products, can be appropriately blended, as needed, into the oil-in-water emulsion cosmetic according to the present invention, within a range not compromising the purpose and effects of the present invention. Such additive components include, for example, polyhydric alcohols, non-ionic surfactants, film agents, astringents, chelators, pH adjusters, antioxidants, various types of medicinal agents such as whiteners, fragrances, etc. However, they are not limited to those mentioned above.

The vesicles that are the precursors of the nanodiscs according to the present invention can be produced as an aqueous vesicle dispersion by well-mixing the (B) polyoxyalkylene-modified silicone with the (A) aqueous component, then dripping the mixture thereof, while stirring, into a water phase containing the aqueous components other than component (A). The mixture state of the (B) polyoxyalkylene-modified silicone and the (A) aqueous component need only be such as to allow one to confinrm that the mixture is in a transparent, single-phase state, which can be achieved, for example, by mixing for 1 to 30 minutes within a temperature range from room temperature to 90° C. Due to this method, vesicle particles having an average particle size of 30 to 150 nm measured by dynamic light scattering can be obtained.

The vesicles according to the present invention can also be produced, by a conventional method, in a form in which oil-based components are held inside the bimolecular films of the vesicles. Specifically, the vesicles according to the present invention may be produced as vesicles in which oil-soluble components, such as fragrances, are held inside bimolecular films of the vesicles by adding and mixing said oil-soluble components during the step of mixing the (B) polyoxyalkylene-modified silicone with the (A) aqueous component.

In the oil-in-water emulsion cosmetic according to the present invention, an oil is added to a water phase containing vesicles and dispersed while applying a strong stirring force, thereby forming and stabilizing a water phase/nanodisc phase/oil phase three-phase structure in which the nanodiscs (phase) that have transitioned from vesicles are adhered to the oil phase (oil droplets).

Thus, the oil-in-water emulsion cosmetic according to the present invention is characterized in that the nanodiscs comprising polyoxyalkylene-modified silicone are adhered (localized) at the oil-water interfaces, i.e., around the oil droplets composed of the oil phase. The nanodiscs have long diameters of 20 nm to 1000 nm.

The water phase/nanodisc phase/oil phase three-phase structure in the oil-in-water emulsion cosmetic of the present invention can be formed by a conventional method. That is, by dripping the polyoxyalkylene-modified silicone into the aqueous component while stirring to form vesicle particles, thereby obtaining an aqueous vesicle dispersion, adding a separately mixed and dissolved oil-based component to this aqueous vesicle dispersion and dispersing with a strong stirring force, the vesicles transition to nanodiscs, and a water phase/nanodisc phase/oil phase three-phase structure is obtained. At this time, in the case in which an anionic surfactant is to be blended, it should preferably be added to the aqueous vesicle dispersion before the oil-based component is added. Since the oil droplets comprising the oil-based component are emulsified and dispersed in the water phase, and the nanodiscs are further localized at the oil droplet particle surfaces, the dispersion has excellent emulsion stability and also has an excellent feel in use (wateriness, lack of stickiness). The stirring device used for stirring is not particularly limited, and for example, a homomixer, a dispersion mixer, etc. may be used.

In the present invention, the vesicle particles formed in the water phase can be shaped to have sufficiently microscopic particle sizes and can be uniformly dispersed in the water phase by applying strong shear with the aforementioned homomixer, etc. Although the level of the strong shear is not particularly limited, it should normally be applied for approximately 5 minutes under conditions of 7000 to 12000 rotations per minute by a homomixer.

In the case in which the (F) anionic surfactant is to be blended in the present invention, it is preferable to form vesicle particles in the water phase, to add the (F) anionic surfactant to the vesicle dispersion, and then to add and emulsify the oil-based component.

Therefore, the method for producing the oil-in-water emulsion cosmetic according to the present invention involves: a vesicle formation step of forming vesicles by mixing the (A) aqueous component with the (B) polyoxyalkylene-modified silicone; in some cases, a step of adding the (F) anionic surfactant to the vesicle dispersion obtained by the vesicle formation step; and an emulsification step of emulsifying the separately mixed and dissolved oil-based component in the mixture obtained by the previous step while stirring and applying a shear force.

In the vesicle formation step, the (A) aqueous component and the (B) polyoxyalkylene-modified silicone may be pre-dissolved and the solution thereof may be mixed into the remaining water phase components to obtain a vesicle dispersion in which vesicles are dispersed in the water phase, or the (B) polyoxyalkylene-modified silicone may be mixed and stirred in a water phase containing the (A) aqueous component and the aqueous components other than component (A) to obtain a vesicle dispersion in which vesicles are dispersed in the water phase.

The cosmetic according to the present invention has a watery texture in use that is characteristic of oil-in-water emulsions, and achieves excellent UV protection effects. Additionally, the nanodisc-containing emulsion of the present invention can obtain a fresh feel in use despite blending large quantities of oils that would not be able to be blended into normal solubilized products.

The cosmetic according to the present invention can be favorably used in various formats, such as in cream form, emulsion form, liquid form, etc. In terms of the product form, it may be provided as a skin-care cosmetic, such as a sunscreen, or a makeup cosmetic, such as a makeup base, a foundation, etc. having covering performance against skin roughness, spots/freckles, etc.

### EXAMPLES

Though the present invention will be explained in further detail by providing examples below, the present invention is not limited in any way by these examples. Where not specially noted, the blended amounts are indicated in percentage by mass relative to the systems in which the relevant components are blended. Before specifically explaining the respective examples, the evaluation methods that were employed will be explained.

### 1. Usage Evaluation

Actual usage tests were performed by five expert panelists. The texture in use (wateriness, lack of powderiness, spreadability when applied) when each cosmetic was applied to the skin was evaluated based on the criteria indicated below.

### <Evaluation Criteria>

A. Responded that the texture in use of the test sample was very good.
B. Responded that the texture in use of the test sample was good.
C. Responded that the texture in use of the test sample was not very good.
D. Responded that the texture in use of the test sample was not good at all.

### Examples

Liquid foundations having the compositions listed in Table 1 were prepared. Specifically, after dripping and mixing component (B) into component (A) while stirring, the other aqueous components and component (F) (only Examples 1, 4 and 5) were mixed to obtain a water-phase solution, and an oil-phase solution separately obtained by mixing the oil-based components and the powder components was mixed into the water-phase solution while stirring, thereby obtaining oil-in-water emulsion cosmetics. The prepared samples were evaluated for stability and texture in use in accordance with the evaluation methods described above. The results are indicated in the table.

TABLE 1

| Category | Components | Ex. 1 | Ex. 2 | Ex. 3 | Ex. 4 | Ex. 5 |
|---|---|---|---|---|---|---|
| | Water | bal | bal | bal | bal | bal |
| Humectant | Glycerin | 7 | 7 | 7 | 7 | 7 |
| | PEG-8 | — | 5 | 5 | — | — |
| Component A | Dipropylene glycol | 5 | 8 | 8 | 8 | 5 |
| | Ethanol | 5 | — | — | 5 | 5 |
| | Modified alcohol | — | 5 | 5 | — | — |
| Water-phase | (Acrylates/alkyl (C10-30) acrylate) | 0.1 | 0.1 | 0.1 | 0.1 | 0.1 |
| Thickener | crosspolymer | | | | | |
| | (Ammonium acryloyl dimethyl taurate/ | 0.3 | 0.3 | 0.3 | 0.3 | 0.3 |
| | beheneth-25 methacrylate) crosspolymer | | | | | |
| | (Dimethylacrylamide/sodium acryloyl | — | 0.1 | 0.1 | — | — |
| | dimethyl taurate) crosspolymer | | | | | |
| Component E | PEG/PPG-19/19 dimethicone | — | 0.00005 | 0.5 | — | — |
| | Disteardimonium hectorite | — | — | — | 0.5 | — |
| | Dextrin palmitate | — | — | — | — | 0.5 |
| Component B | PEG-12 dimethicone | 1.6 | 1 | 1 | 1.6 | 1.6 |
| Component F | Sodium stearoyl methyl taurate | 0.01 | — | — | 0.01 | 0.01 |
| | PEG-60 hydrogenated castor oil | 1.2 | 1.5 | 1.5 | 1.2 | 1.2 |
| | PEG-100 hydrogenated castor oil | 0.5 | 0.7 | 0.7 | 0.5 | 0.5 |
| Component G | Isostearic acid | 1 | 1 | 1 | 1 | 1 |
| | Polyglyceryl-6 polyricinoleate | 1 | 1 | 1 | 1 | 1 |
| Neutralizer | Potassium hydroxide | 0.04 | 0.05 | 0.05 | 0.04 | 0.04 |
| Component C | Ethyl hexyl methoxycinnamate | 7 | 7 | 7 | 7 | 7 |
| | Diphenyl siloxyphenyl trimethicone | 9 | 9 | 9 | 9 | 9 |
| | Hydrogenated polyisobutene | 1 | — | — | 1 | 1 |
| Medicinal | Niacin amide | 5 | 5 | 5 | 5 | 5 |
| Agent | Tocopherol acetate | 0.1 | 0.1 | 0.1 | 0.1 | 0.1 |
| | Lactobacillus/fermented rice | 0.7 | 0.7 | 0.7 | 0.7 | 0.7 |
| Component D | Amino acid-treated titanium oxide | 7.35 | 7.35 | 7.35 | 7.35 | 7.35 |
| | Amino acid-treated yellow iron oxide | 0.56 | 0.56 | 0.56 | 0.56 | 0.56 |
| | Amino acid-treated red iron oxide | 0.16 | 0.16 | 0.16 | 0.16 | 0.16 |
| | Amino acid-treated black iron oxide | 0.15 | 0.15 | 0.15 | 0.15 | 0.15 |
| Preservative | Phenoxyethanol | s.a. | s.a. | s.a. | s.a. | s.a. |
| Chelator | Trisodium EDTA | 0.05 | 0.1 | 0.1 | 0.05 | 0.05 |
| | Total | 100.0 | 100.0 | 100.0 | 100.0 | 100.0 |
| Evaluation | Wateriness | A | A | A | A | A |
| | Lack of powderiness | A | A | A | A | A |
| | Lightness to spread | A | A | A | A | A |

As indicated in Table 1, the cosmetic of Example 1, in which components (A) to (D) of the present invention were blended, and the cosmetics of Examples 2 to 5, in which components (A) to (E) were blended, all were watery, did not have powderiness, and had excellent spreadability when applied.

Additionally, each prepared sample was put in a 50-ml sample tube (glass vial) and the outer appearance after being placed at rest for 1 hour was observed by eye to evaluate the powder dispersion stability, whereupon the cosmetics of Examples 2 to 5 including component (E) in the present invention were less susceptible to color streaking than the cosmetic of Example 1, which did not include component (E). This demonstrated that blending component (E) improved the powder dispersibility.

1. An oil-in-water emulsion cosmetic containing:
(A) an aqueous component selected from monohydric alcohols and dihydric alcohols;
(B) a polyoxyalkylene-modified silicone;
(C) an oil; and
(D) a pigment having a hydrophobized surface;
wherein the (A) aqueous component, in the case of being a monohydric alcohol alone, constitutes 1% to 15% by mass relative to the overall amount of the cosmetic, in the case of being a dihydric alcohol alone, constitutes 1% to 20% by mass relative to the overall amount of the cosmetic, and in the case of being a combination of a monohydric alcohol and a dihydric alcohol, a total amount thereof constitutes 1% to 45% by mass relative to the overall amount of the cosmetic.

2. The oil-in-water emulsion cosmetic according to claim 1, wherein a percentage by mass of the (C) oil occupied by a silicone oil is 50% or higher.

3. The oil-in-water emulsion cosmetic according to claim 1, wherein the component (D) is hydrophobically treated by a surface treatment agent containing an amino acid.

4. The oil-in-water emulsion cosmetic according to claim 1, wherein the component (B) is PEG-12 dimethicone.

5. The oil-in-water emulsion cosmetic according to claim 1, further containing (E) an oil-phase thickener.

6. The oil-in-water emulsion cosmetic according to claim 1, further containing (F) an anionic surfactant.

7. The oil-in-water emulsion cosmetic according to claim 1, further containing (G) a dispersant.

8. The oil-in-water emulsion cosmetic according to claim 7, wherein the component (G) is a polyglycerin fatty acid ester having three or more glycerin molecules.

9. The oil-in-water emulsion cosmetic according to claim 1, wherein nanodiscs comprising the component (B) are adhered to an oil-water interface.

10. The oil-in-water emulsion cosmetic according to claim 1, which is a makeup cosmetic.
